(19) United States
(12) Patent Application Publication
(10) Pub. No.: US 2009/0311719 A1
(43) Pub. Date: Dec. 17, 2009

(54) IN VITRO METHOD FOR DIAGNOSING NEURODEGENERATIVE DISEASES

(75) Inventors: Andreas Bergmann, Berlin (DE); Joachim Struck, Berlin (DE); Andrea Ernst, Hennigsdorf (DE)

Correspondence Address: HESLIN ROTHENBERG FARLEY & MESITI PC, 5 COLUMBIA CIRCLE, ALBANY, NY, 12203, US

(73) Assignee: B.R.A.H.M.S AKTIENGESELLSCHAFT, Hennigsdorf (DE)

(21) Appl. No.: 12/091,261

(22) PCT Filed: Oct. 26, 2006
(86) PCT No.: PCT/EP06/10352

§ 371 Date: Apr. 23, 2008

(30) Foreign Application Priority Data
Oct. 26, 2005 (EP) 05023420.2

(51) Int. Cl. C12Q 1/25

(52) U.S. Cl. 435/7.4

(57) ABSTRACT

Disclosed is an in vitro method for the detection, for the determination of the severity and for the assessment of the progress and prediction of neurodegenerative diseases, in which the presence and/or concentration of carbamoyl phosphate synthetase 1 (CPS 1) is determined in a biological fluid of a patient who suffers from a neurodegenerative disease or is suspected of suffering from such a disease, and conclusions about the presence, progression, severity or success of a treatment of the neurodegenerative disease are drawn on the basis of the determined presence and/or concentration of CPS 1 or the non-detectability of a CPS 1 immune reactivity.

### FIELD OF THE INVENTION

The present invention relates to a novel in vitro method for diagnosing neurodegenerative diseases, in particular dementias, such as Alzheimer's disease. The invention is based on the surprising discovery that an enzyme from the liver, namely the enzyme carbamoyl phosphate synthetase 1 (E.C. 6.3.4.16, always abbreviated to CPS 1 below), and/or physiologically occurring CPS 1 fragments having CPS 1 immune reactivity is found at significantly increased levels in the circulation of Alzheimer patients, in particular in plasma or serum, and is therefore suitable for use as a humoral biomarker in medical diagnosis for the detection of neurodegenerative diseases.

### BACKGROUND OF THE INVENTION

In the present invention, terms such as “diagnosis” or “diagnostic” are used as general terms for medical determinations which, depending on the clinical condition of the patient for whom the determination is carried out and depending on the information already available about him, may be based on different problems and which serve in particular for the detection and early detection, the determination of the severity and the assessment of the progression, including the assessment of the progression during treatment, and the prediction of the future progression of a disease.

Of particular importance in the present context is that a diagnosis may also be a negative diagnosis in which the presence of a certain disease is reliably excluded on the basis of the non-determinability of a certain feature typical of the disease, for example the non-detectability of a biomarker associated with the relevant disease in the blood sample of a patient.

Biomarkers which can be found at an increased level in a plurality of different diseases and therefore do not by themselves permit a positive diagnosis of a specific disease—although they may as a rule be decisive also for the positive diagnosis on inclusion of further clinical or biochemical parameters—are also of great value for the negative diagnosis.

The diseases with the diagnosis of which the present invention is concerned tend to be slowly developing, chronic neurodegenerative diseases of non infectious aetiology, in particular presenile dementias.

Dementias are generally defined as diseases for which a common feature is the loss of acquired intellectual abilities, especially of memory, and of the normal level of personality as a result of brain damage. Dementias are as a rule relatively slowly developing diseases of chronic character. If dementia symptoms occur before old age, in middle age, they are referred to as presenile dementias, and among these a distinction is made on the basis of the symptoms and cerebropathological changes typical of them, in particular the following diseases or groups of diseases:

Alzheimer's Disease (AD) is the most frequent neurodegenerative dementia and accounts for ⅔ of all cases of dementia. AD is distinguished by three important pathological features which, however, can only be established with certainty post mortem: the formation of amyloid plaques and neurofibrillar bundles and the loss of nerve cells (for overview cf. SELKOE D. J. (2001). Alzheimer's disease: genes, proteins, and therapy. Physiological Reviews 81: 741-766). Amyloid plaques consist of extraneuronal aggregates of amyloid β protein, while the neurofibril bundles contain mainly tau-protein and neurofilaments. It is presumed that the plaque and neurofibril formation causes nerve cells to die.

The most important symptoms of AD are increasing disturbances of memory and of thought processes with relatively persistent emotional responsiveness, these systems being accompanied by further less specific disturbances which make it difficult to differentiate AD from other forms of dementia.

Dementia with Lewy bodies (DLB) is a second most frequent cause of a dementia after Alzheimer's disease. Neuropathalogically, DLB is characterized by the occurrence of so-called Lewy bodies in the brain stem and in the cortex. These Lewy bodies predominantly comprise aggregates of the presynaptic protein (α-synuclein) and ubiquitin. The Lewy body pathology may be associated to different extents with neuropathalogical changes typical of Alzheimer's and Parkinson's disease. Thus, in DLB too, the formation of beta-amyloid and senile plaques occurs, but not neurofibril bundles (for an overview, cf. MCKEITH I. G. (2002). Dementia with lewy bodies. British Journal of Psychiatry 180: 144-147; cf. also GELDMACHER D. S. (2004). Dementia with Lewy bodies: diagnosis and clinical approach. Cleveland clinic Journal of Medicine 71:789-800). Lewy bodies are also present in the brain of patients with Parkinson's disease, even if in a different distribution.

Key symptoms of DLB are a progressive cognitive disturbance, episodes of confusion with fluctuating attention and consciousness, Parkinsonism, frequent falls and syncopes (brief, paroxysmal unconsciousness). The sensitivity and specificity of the diagnostic criteria show high specificity throughout but a very low sensitivity in some cases. This means that DLB is frequently not diagnosed in clinical routine.

Frontotemporal Dementia (FTD) is also referred to as Pick's disease and accounts for about 20% of presenile dementias. FTD is genetic in some cases and is among the so-called tauopathies, which are distinguished by overexpression or underexpression of a tau-protein subtype or by expression of a mutated tau-protein. Neuropathalogical symptoms are local atrophy of the frontal and/or temporal cortex and of the substantia nigra and of the basal ganglia. This results in different levels of speech disturbance, a change of personality and behavioural peculiarities. Overall, FTD is underdiagnosed with a sensitivity of 93% at a specificity of only 23%, AD being the most frequent misdiagnosis.

The term vascular dementia (VAD) covers diseases in which a dementia is triggered owing to disturbed blood flow in the brain. There are different types of VAD, of which multi-infarction dementia (MID) and subcortical VAD (also referred to as Binswanger's disease) are the most frequent forms.

Binswanger's disease is a slowly progressing dementia which is characterized pathologically by cerebrovascular lesions in the white brain substance. Clinically, this results in behavioural peculiarities, such as agitation, irritability, depression and euphoria, and slight memory disturbance.

Multi-infarction dementia arises gradually as a consequence of several small strokes, also referred to as transient ischaemic attacks (TIA), which led to the destruction of brain tissue in the cortex and/or subcortical areas. The strokes may also have remained completely unnoticed, in which case the dementia is the first noticeable consequence. In the presence of MID, there is a gradual decrease in cognitive abilities, associated with severe depressions, mood fluctuations and epilepsy.

A diagnosis on dementias is performed nowadays predominantly on the basis of neuropsychological investigations and the observation of the development of the disease and its progress, using exclusion criteria for certain forms of dementia. In very many cases, these investigations give ambiguous results, which explain the above mentioned numbers for the underdiagnosed forms of dementia or incorrectly diagnosed cases. The cerebral changes typical of the disease cannot of course be directly established in living patients and technical medical investigations of brain functions by means of, for example, Xray tomography or MRI are complicated and expensive.

For the diagnosis of Alzheimer's disease, the Ronald and Nancy Reagan Institute of the Alzheimer Association and the NIA Working Group published guidelines for the criteria which are set with regard to an ideal biomarker for detecting AD (7). The following criteria should ideally be fulfilled by the biomarker:

1. It should be brain-specific and detect a fundamental feature of the neuropathology of these diseases.

2. There should be diagnostic sensitivity and specificity of at least 80%.

3. The disease-specific change of the biomarker should manifest itself at as early a stage of the disease as possible in order to be able to begin suitable therapeutic measures (GROWDON J. H., SELKOE D. J., ROSES A., TROJANOWSKI J. Q., DAVIES P., APPEL S. et al. [Working Group Advisory Committee].(1998). Consensus report of the Working Group on Biological Markers of Alzheimer's Disease. [Ronald and Nancy Reagan Institute of the Alzheimer's Association and National Institute on Aging Working Group on Biological Biomarkers of Alzheimer's Disease]. Neurobiology of Aging 19: 109-116).

To date, however, there is no biomarker which could be used in clinical routine in the blood or the cerebrospinal fluid with sufficient safety for early and differential diagnosis of AD and which fulfils all abovementioned criteria. At present, various potential marker candidates are being investigated, including information markers, such as IL-6 and TNFα, markers for oxidative stress, such as 3-nitrotyrosine, and markers which are associated with characteristic pathological changes of AD, such as amyloid β, which is a main constituent of amyloid plaques, and the tau-protein, which is a substantial constituent of the neurofibril bundles (cf. the overviews in FRANK R. A., GALASKO D., HAMPEL H., HARDY J., DE LEON M. J., MEHTA P. D., ROGERS J., SIEMERS E., TROJANOWSKI J. Q. (2003). Biological markers for therapeutic trials in Alzheimer's disease. Proceedings of the biological markers working group; NIA initiative on neuroimaging in Alzheimer's disease. Neurobiology of Aging 24: 521-536; or in TEUNISSEN C. E., DE VENTE J., STEINBUSCH H. W. M., DE BRUIJN C. (2002). Biochemical markers related to Alzheimer's dementia in serum and cerebrospinal fluid. Neurobiology of Aging 23:485-508).

WO 2003/089934 furthermore discloses that the peptide LASP-1, which is also found in the circulation of sepsis patients, is significantly increased in serum and in plasma of Alzheimer patients. The clinical value of this finding is currently the subject of further investigations.

There continues at present to be an actual need for supplementary investigative methods which give valid laboratory findings and are based on a determination of substances suitable as biomarkers for dementias, in particular for Alzheimer's disease (AD), in blood or plasma samples and are suitable for a positive diagnosis and/or for a negative exclusion diagnosis in patients who are suspected of suffering from a dementia, in particular from AD.

The present invention provides such an investigative method in the form of an in vitro method for the detection, for the determination of the severity and for the assessment of the progress and prediction of neurodegenerative diseases, which is characterized in that the presence and/or concentration of the enzyme carbamoyl phosphate synthetase 1 (CPS 1) and/or physiologically occurring CPS 1 fragments having CPS 1 immune reactivity is determined in a biological fluid of a patient who suffers from a neurodegenerative disease or is suspected of suffering from such a disease, and conclusions about the presence, progression, severity or success of a treatment of the neurodegenerative disease are drawn on the basis of the determined presence and/or concentration of CPS 1 or the non-detectability of a CPS 1 immune reactivity.

Advantageous or preferred developments of the method according to claim 1 are reproduced in subclaims 2 to 12.

The present invention is based on completely surprising findings of a systematic investigation of a large number of sera and plasmas of patients with a diagnosis “probably Alzheimer's disease” with the aid of novel immunoassays for determining various biomarkers, which are undergoing development and clinical trials by the applicant and in some cases form the subject of published or still unpublished earlier patent applications of the applicant.

The results of measurements in EDTA plasma samples of apparently healthy normal persons and Alzheimer patients, described below in the experimental section, showed a clear, diagnostically significant correlation between the concentrations found for CPS-1 and the presence of dementia symptoms which had led to the diagnosis “probably Alzheimer”.

Although the investigations were limited to date to plasma samples of Alzheimer patients, the inventors assume that—possibly with different typical concentration ranges—characteristic increases in CPS-1 concentrations would also have to be detectable in patient plasmas in the case of other neuro inflammatory dementia forms, in particular in vascular dementia (VAD) and dementia with Lewy bodies (DLB).

The assay method for CPS-1 in patient plasmas used for the measurements described in the experimental section, is a modification of a non competitive immunoluminometric sandwich assay for determining CPS-1, as described in more detail in WO 03/089933 or EP 1 497 662 A1 of the applicant.

For medical diagnosis, CPS 1 and CPS 1 fragments having CPS 1 immune reactivity traditionally played no practical role. In the area of the diagnosis of neurodegenerative diseases, the liver enzyme CPS 1 has as yet never been considered as a possible humoral biomarker.

The enzyme CPS 1 (E.C. 6.3.4.16) itself has, however, long been well known. It catalyses the conversion of ammonia, bicarbonate and 2 ATP with formation of carbamoyl phosphate in the first step of the urea cycle. It also plays a role in the biosynthesis of arginine, which in turn is a substrate for the biosynthesis of NO, e.g. in the case of an endotoxin shock (cf. Shoko Tabuchi et al., Regulation of Genes for Inducible Nitric Oxide Synthase and Urea Cycle Enzymes in Rat Liver in Endotoxin Shock, Biochemical and Biophysical Research Communications 268, 221-224 (2000)). CPS 1 should be distinguished from the cytosolic enzyme CPS 2 (E.C. 2.7.2.5.), which likewise plays a role in the urea cycle but processes the substrate glutamine. It is known that CPS 1 is localised in mitochondria and occurs in this form in large amounts in liver tissue (it accounts for 2-6% of the total liver protein). Its amino acid sequence and genetic localisation has long been known (cf. Haraguchi Y. et al, Cloning and sequence of a cDNA encoding human carbamyl phosphate synthetase I: molecular analysis of hyperammonemia, Gene 1991, Nov. 1; 107 (2):335-340; cf. also the publication WO 03/089933 A1 of the applicant). Regarding its physiological role, reference may be made to review articles such as, for example H. M. Holder et al., Carbamoyl phosphate synthetase: an amazing biochemical odyssey from substrate to product, CMLS, Cell. Mol. Life Sci. 56 (1999) 507-522 and the literature referred to therein, and the introduction of the publication Mikiko Ozaki et al., Enzyme-Linked Immunosorbent Assay of Carbamoylphosphate Synthetase I: Plasma Enzyme in Rat Experimental Hepatitis and Its Clearance, Enzyme Protein 1994, 95:48:213-221.

The measurements described in the present application constitute the first report on the occurrence of CPS 1 in the circulation of patients with neurodegenerative diseases, in particular patients diagnosed with Alzheimer's disease. To date, CPS 1 was determined, and only in investigations by the applicant, only in serum or plasma of sepsis patients (cf. WO 03/089933 A1). Sepsis patients whose highly acute potentially life-threatening disease is typically monitored and treated in intensive care wards are a patient population clearly differing from patients with neurodegenerative diseases who are suffering from a disease developing over long periods.

In the determination of CPS 1 or CPS 1 immune reactivity as a humoral biomarker in patient sera according to the present invention, it is possible in principle to proceed as described in the publication WO 03/089933 A1 of the applicant in relation to the determination of CPS 1 as a sepsis marker. The assay method which is described in the experimental section of the present application and was used for testing sera or plasmas of patients with a diagnosis “probably Alzheimer's disease” for the presence of CPS 1 or CPS 1 immune reactivity is a modification of the method which was described in the abovementioned application WO 03/089933 A1. The modification takes into account the fact, disclosed in WO 03/089933 A1, that the species to be found in the circulation and having CPS 1 immune reactivity is itself the complete, or at least substantially complete, enzyme CPS 1. In addition to a first antibody binding to the amino acids 184-199 of the amino acid sequence of human CPS 1, as also used in the abovementioned WO 03/089933, a second antibody which binds to the amino acids 781 to 794 of said sequence is used.

In the context of the present application, not only the direct immunological determination of CPS 1 in in vitro samples for diagnostic purposes but also a use of CPS 1 or CPS 1 fragments, or of antibodies for their selective determination, for the production of assay kits, or a use for the production of assay components, e.g. of polyclonal or monoclonal antibodies which are provided, for example, in immobilised or marked form as a rule likewise in assay kits for said diseases, or of standard and reference substances, is to be regarded as “use of CPS 1 as a biomarker”.

It is additionally expressively pointed out that, in the determination according to the invention of CPS 1 or CPS 1 immune reactivity, a simultaneous determination of CPS 1 both in the form of the substantially complete molecule and in the form of other, shorter fragments (physiologically occurring partial peptides) of the complete CPS 1, possibly present in the biological fluid, can be effected depending on the assay design. Where a determination of “CPS 1 immune reactivity” is mentioned in the present application, it is intended to take into account this circumstance with respect to measurement technology, so that an improper confining interpretation of the teaching of the present invention is avoided.

Instead of the determination of CPS 1 or CPS 1 immune reactivity, for diagnostic purposes the CPS 1 determination should also be capable, if appropriate, of being effected indirectly as a determination of an enzyme activity which corresponds to the CPS 1 activity or the residual activity of the CPS 1 fragments in the blood. Since CPS 1 does not occur in the circulation in healthy persons, a measurable CPS 1 enzyme activity in the blood of a patient may be a diagnostically significant indication of a serious disturbance of the sound health of the patient. It should also be pointed out here that the activity of an enzyme which is usually localised in the interior of the cell and displays its proper functional effect only there, is to be regarded as being negative per se in the circulation and can therefore also contribute as such to an escalation of a pathological condition.

The actual CPS 1 determination can be effected not only in the manner specifically described in the experimental section but in any suitable manner known per se, immunoassays of a suitable assay design being preferred.

The methods for determining CPS 1 immune reactivity in a biological sample may be any known immunodiagnostic methods which are used for detecting and for measuring antigens. Preferably, CPS 1 is determined with the aid of a ligand-binding assay in which specific antibodies suitable for binding and marking are used in immobilised form or marked or markable form.

Competitive assay formats may also have particular advantages. Instead of employing enzyme marking, another marking is preferably chosen, for example marking for a chemiluminescence detection reaction, e.g. an acridinium ester. Of course, it is preferable to use for the CPS 1 determination an assay which ensures the required high sensitivity in the range of the CPS 1 concentrations occurring in neurodegenerative diseases and permits separation of the measurement signals from the assay background.

The assay method can be adapted to the chip technology or developed as an accelerated test (point-of-care-test).

In a preferred embodiment, the immunodiagnostic determination is carried out as a heterogeneous sandwich immunoassay in which one of the antibodies is immobilised on any solid phase, for example the walls of coated test tubes (e.g. of polystyrene; “Coated Tubes”; CT) or on microtitre plates, for example of polystyrene, or on particles, for example magnetic particles, while the other antibody carries a residue which is a directly detectable label or permits selective linking to a label and serves for the detection of the sandwich structures formed. Delayed or subsequent immobilisation with the use of suitable solid phases is also possible.

In principle, all marking techniques which can be used in assays of the type described can be employed, including markings with radioisotopes, enzymes, fluorescent, chemoluminescent or bioluminescent labels and directly optically detectable colour markings, such as, for example, gold atoms and dye particles, as are used in particular for so-called point-of-care (POC) or accelerated tests. In the case of heterogeneous sandwich immunoassays, the two antibodies may also have parts of a detection system of the type described below in relation to homogeneous assays.

The method according to the invention can furthermore be designed as a homogeneous method in which the sandwich complexes formed from the two antibodies and the CPS 1 to be detected remain suspended in the liquid phase. In such a case, it is preferable to mark both antibodies with parts of a detection system which, if both antibodies are integrated in a single sandwich, then permits signal generation or signal triggering. Such techniques can be designed as particular as fluorescence amplification or fluorescence extinction detection methods. A particularly preferred method of this type relates to the use of detection reagents to be used in pairs, as described, for example, in U.S. Pat. No. 4,822,733, EP-BI-180 492 or EP-BI-539 477 and the prior art cited therein. They permit a measurement which selectively determines only reaction products which contain both marking components in a single immune complex, directly in the reaction mixture. The technology available under the brands TRACE® (Time Resolved Amplified Cryptate Emission) or KRYPTOR®, which implements the teachings of the abovementioned applications, may be referred to as an example.

The content of said prior application (WO 03/089933 A1) of the applicant is to be regarded as part of the disclosure of the present application by express reference to these applications.

### BRIEF DESCRIPTION OF THE DRAWINGS

Below, the invention is explained in more detail with reference to results of measurements and two figures.

FIG. 1 shows a typical calibration curve for the determination of CPS-1 in patient plasmas by the assay described in more detail in the experimental section;

FIG. 2 shows the results of the measurement of the CPS-1 concentrations and EDTA plasmas of patients with a diagnosis “probably Alzheimer's disease” (prAD), compared with the results of the same measurements in the case of age-matched control persons without symptoms for Alzheimer's disease.

### EXPERIMENTAL SECTION

### Assay Description

### Preparation of the Antibodies

a) Immunogens

Two different peptide sequences were chosen from the complete sequence of human CPS 1, in particular a first peptide sequence 1 (EFEGQPVDFVDPNKQN), which corresponds to the amino acids 184-199 of the sequence of human CPS 1 (cf. Peptide PCEN17 according to WO 03/089933), and a second peptide sequence (FHGTSSRIGSSMKS), which corresponds to the amino acids 781-794 of the sequence of human CPS 1. Each peptide was synthesised by Jerini (Berlin, Germany) in a form provided with an amino-terminal cysteine residue (Cys0). The synthesised peptides used for the following immunisations are shown as SEQ ID NO:1 and SEQ ID NO:2, respectively, in the sequence listing.

b) Antibodies

For the immunisation, the two synthesised peptides were conjugated with haemocyanine from Limulus polyphemus, and, as also described in WO 03/089933, polyclonal antibodies were produced in sheep by Micropharm Ltd. (Carmarthenshire, UK).

2. Purification of the Antibodies

The antibodies were purified by means of ligand-specific affinity purification. For this purpose, Cys(0) peptides 1 and 2 were first coupled to SulfoLink gel from Pierce (Boston, USA). The binding was effected according to the manufacturer's instructions.

The procedure was as follows: polycarbonate columns (15 mm×80 mm) were filled with 5 ml of affinity matrix. After equilibration of the columns with PBS (136 mM NaCl, 1.5 mM KH₂PO₄, 20.4 mM Na₂HPO₄·2H₂O, 2.7 mM KCl, pH 7.2), 5 mg of the respective peptides were weighed out, dissolved in PBS and added to the closed columns. The gel material was homogenised by swirling.

After incubation for 15 minutes at room temperature and settling of the gel material, the columns were washed with five 3 ml portions of PBS. For saturating free binding sites, 5 ml of a 50 mM L-cysteine solution were added in each case to the column material and, after homogenisation, the gel material was incubated again at room temperature for 15 min. After settling of the gel material, each column was washed six times with 5 ml of a 1M NaCl solution and then washed again with PBS.

The gel material was mixed with 25 ml of the respective antiserum pool and incubated overnight at room temperature with gentle swirling. The serum-gel mixtures were transferred to polycarbonate columns, and excess serum was removed. The columns were then washed with 250 ml of PBS in order to remove serum proteins which were not bound. The desorption of the bound antibodies was effected by elution of the column with 50 mM citric acid (pH 2.2). The eluate was collected in 1 ml fractions. The protein concentration of each fraction was determined with the aid of the BCA protein assay kit from Perbio (Bonn, Germany), and the fractions with a protein content >1 mg/ml were combined. The affinity-purified antibodies were rebuffered by means of dialysis in PBS and, after determination of the protein content again, then stored 4° C.

3. Immobilisation/Marking of the Antibodies

The purified antibodies against the peptide which corresponds to the amino acid sequence 781-794 were immobilised on polystyrene tubes (Startubes, 12 mm×75 mm, from Greiner, Germany). For this purpose, the antibody solutions were diluted to a protein concentration of 6.7 μg/ml with PBS and 300 μl were pipetted per tube (corresponds to 2 μg of antibody per tube). These were incubated for 20 h at room temperature and then washed 3 times with 4 ml portions of PBS. The tubes were stored at 4° C. until required for further use.

The antibody against the peptide which corresponds to the amino acid sequence 184-199 (1 mg/ml in PBS) was luminescence-marked with acridinium ester N-hydroxysuccinimide (1 mg/ml in acetonitrile, from InVent, Hennigsdorf, Germany). For the marking, 200 μl of antibody were mixed with 4 μl of acridinium ester and incubated for 20 min, and free acridinium ester bonds were saturated by addition of 40 μl of a 50 mM glycine solution. The marking batch was separated from free acridinium ester by means of HPLC on a BioSil 400 gel filtration column (from BioRad, Munich, Germany). The mobile phase used was PBS.

4. Relative Calibration

In order to be able to determine the relative CPS 1 concentrations, a pool of plasmas from human patients with SIRS with particularly high CPS 1 concentrations was used as standard material. The CPS 1 concentration of this pool was set arbitrarily at 150 U/ml. Starting from this pool, standards to which arbitrary concentrations were ascribed according to the dilution were prepared by serial dilution with CPS 1-free human plasma from healthy persons.

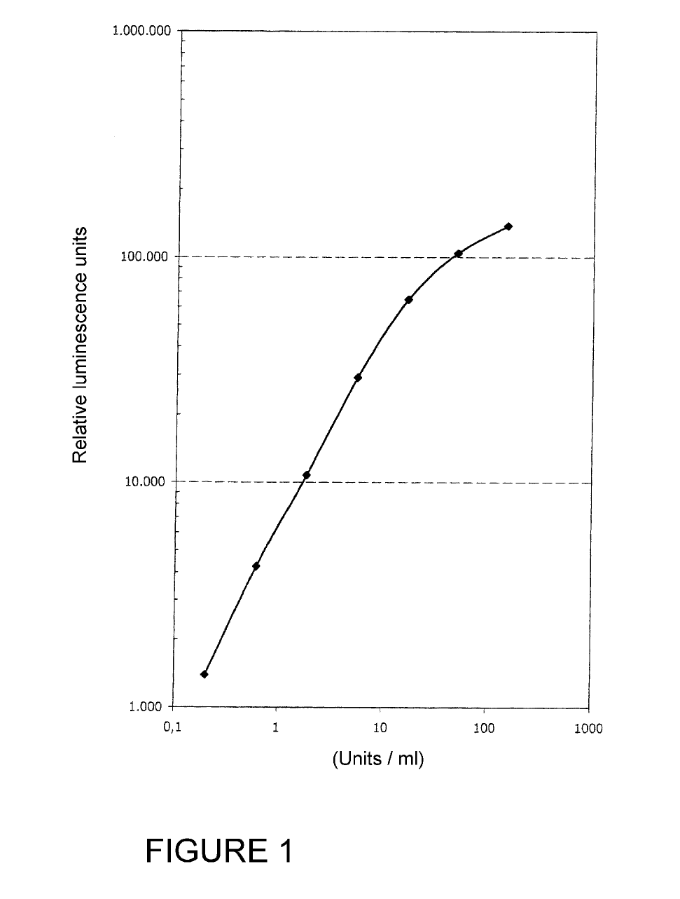

A typical standard curve with the corresponding relative concentrations is shown in FIG. 1. The analytical assay sensitivity is 0.9 U/ml.

5. Assay Procedure

50 μl of a plasma sample and 100 μl of PBS buffer (with 10 mM EDTA) were pipetted per antibody-coated tube and incubated for 16 h at room temperature. After washing 3 times with 1 ml portions of PBS 15 ng of the marked antibody (in 200 μl of PBS buffer, 10 mM EDTA) were added per tube. The tubes were incubated for a further 2 h, and unbound tracer antibody was then removed by washing 5 times with 1 ml portions of PBS. Marked antibody bound to the tube was quantified by means of luminescence measurement in a luminometer (Berthold LB 952T/16).

Measurement of CPS 1 in the Plasma of Healthy Controls and Patients with Probable Alzheimer's Disease

Using the assay described above, relative CPS 1 concentrations in 115 EDTA plasma samples of apparently healthy control persons and in 105 EDTA plasma samples of patients with probable Alzheimer's disease (prAD patients; clinically diagnosed on the basis of the NINCDS/ARDA criteria, cf. McKhann G., Drachman G., Folstein M., Katzman R., Price D., Stadlan E. M.: Clinical diagnosis of Alzheimer's disease: Report of the NINCDS/ARDA workgroup under the auspices of the Department of health and human services task force on Alzheimer's disease, Neurology 34:939-944 (1984)) were determined in the stated manner.

The controls have a median age of 67.9±12.4 years and the prAD patients a median age of 73.3±9.4 years.

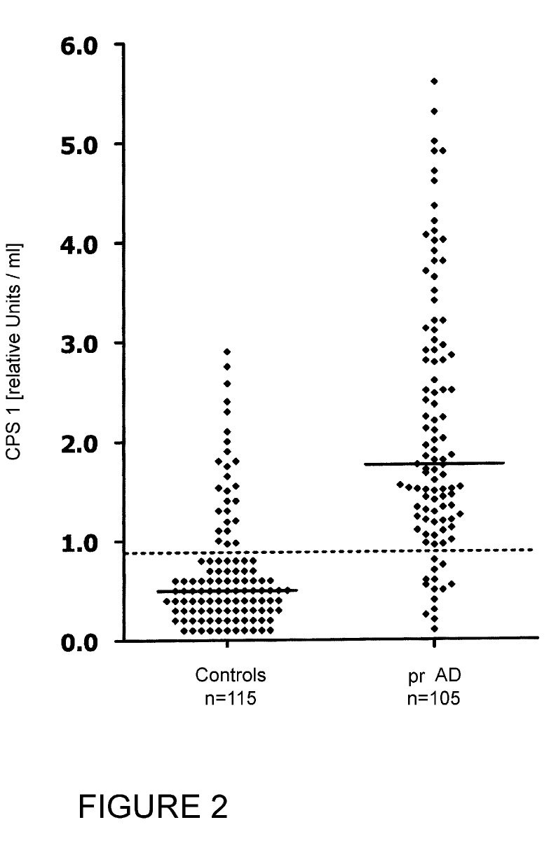

The cut-off of the assay was set at 0.9 U/ml (relative units per ml), which corresponds to the analytical sensitivity of the assay. In 91 out of 105 prAD patients, relative CPS 1 concentrations of more than 0.9 U/ml were measured. In contrast, the measurable CPS 1 concentrations of 89 out of 115 controls are below 0.9 U/ml. The results are shown graphically in FIG. 2.

Thus, Alzheimer patients can be distinguished from controls with a specificity of 77.4% and a sensitivity of 86.7%. The difference between the two groups is highly significant (P value <0.00001). The medial concentration of the controls was below the sensitivity of the test, whereas Alzheimer patients had a medial CPS 1 concentration of 1.76 U/ml.

1. An in vitro method for the detection, for the determination of the severity and for the assessment of the progress and prediction of neurodegenerative diseases, wherein the presence and/or concentration of carbamoyl phosphate synthetase 1 (CPS 1) and/or physiologically occurring CPS 1 fragments having CPS 1 immune reactivity is determined in a biological fluid of a patient who suffers from a neurodegenerative disease or is suspected of suffering from such a disease, and conclusions about the presence, progression, severity or success of a treatment of the neurodegenerative disease are drawn on the basis of the determined presence and/or concentration of CPS 1 or the non-detectability of a CPS 1 immune reactivity.

2. The method according to claim 1, wherein the assay method is an immunodiagnostic assay method.

3. The method of claim 1, wherein the immunodiagnostic assay method is an immunoassay of the sandwich type.

4. The method according to claim 3, wherein CPS 1 is determined in the plasma of a patient with the aid of a sandwich assay which detects CPS 1 and CPS 1 fragments which have at least the amino acids 184 to 794 of human CPS 1.

5. The method according to claim 1, wherein the CPS 1 determination is effected as a determination of the CPS 1 enzyme activity in blood, plasma or serum.

6. The method of claim 1, wherein the neurodegenerative disease is a presenile dementia selected from the group consisting of Alzheimer's disease (AD), dementia with Lewy bodies (DLB), frontotemporal dementia (FTD) and various forms of vascular dementia (VD).

7. The method according to claim 6, wherein said method is carried out in the diagnosis of Alzheimer's disease.

8. The method of claim 1, wherein said method is carried out in a multi parameter determination in which at least one further biochemical or physiological parameter informative for the respective clinical picture is determined simultaneously and in which a measured result in the form of a set of at least two measured quantities is obtained, which is evaluated for the fine diagnosis of the neurodegenerative disease.

9. The method according to claim 8, wherein, in the multi parameter determination, at least one further biochemical parameter which is selected from the group consisting of the inflammation mediators, complement components, cytokines, chemokines, blood coagulants and fibrinolytic factors, acute-phase proteins and free radical compounds is determined in addition to the determination of the CPS 1.

10. The method according to claim 8, wherein the peptide LASP-1, a physiologically inactive preproadrenomedullin partial peptide, apolipoprotein A1, apolipoprotein E4 and/or Cu/Zn superoxide dismutase is determined as at least one further biochemical parameter.

11. The method according to claim 8, wherein the multi parameter determination is effected as a simultaneous determination by means of a chip technology measuring apparatus or of an immunochromatographic measuring apparatus.

12. The method of claim 8, wherein the evaluation of the complex measured result of the multi parameter determination is effected with the aid of a computer program.

13. A method for the detection of neurodegenerative disease, said method comprising determining the level of carbamoyl phosphate synthetase 1 (CPS 1) in a biological sample from a patient in whom a neurodegenerative disease is present or suspected with a sandwich assay that uses a first antibody directed to a peptide having the amino acid sequence of SEQ ID NO.: 1 and a second antibody directed to a peptide having the amino acid sequence of SEQ ID NO.: 2; and comparing said level of CPS 1 with the level of CPS 1 in healthy individuals, wherein an elevated level of CPS 1 in comparison to healthy individuals is indicative of neurodegenerative disease.

14. The method of claim 13, wherein said neurodegenerative disease is Alzheimer's Disease.

15. The method of claim 13, wherein a CPS 1 level >0.9 U/ml is indicative of neurodegenerative disease.


SEQUENCE LISTING (2 sequences)

SEQ ID NO: 1 - protein, 17 aa, Artificial
  misc_feature 2..16: Synthetic peptide corresponding to amino acids 184-199 of human carbamoyl phosphate synthetase 1 (CPS1)
CEFEGQPVDFVDPNKQN

SEQ ID NO: 2 - protein, 15 aa, Artificial
  misc_feature 2..15: Synthetic peptide corresponding to amino acids 781-794 of human carbamoyl phosphate synthetase 1 (CPS1)
CFHGTSSRIGSSMKS